(19) United States
(12) Patent Application Publication
(10) Pub. No.: US 2021/0113424 A1
(43) Pub. Date: Apr. 22, 2021

(54) APPARATUS FOR MANUAL HAPTIC STIMULATION OF MALE ORGAN

(71) Applicant: Andrew Yates, Austin, TX (US)

(72) Inventor: Andrew Yates, Austin, TX (US)

(21) Appl. No.: 17/069,465

(22) Filed: Oct. 13, 2020

Related U.S. Application Data
(60) Provisional application No. 62/924,148, filed on Oct. 21, 2019.

(51) Int. Cl. A61H 19/00

(52) U.S. Cl. CPC A61H 19/32; A61H 2201/0157; A61H 2201/1692; A61H 2201/1602; A61H 2201/1253

(57) ABSTRACT

Apparatus for manual haptic stimulation of the male sex organ of an individual may include a penis pump assembly and a traveling carrier assembly. The apparatus also may include a stroker sleeve carried by the travelling carrier assembly. The traveling carrier assembly may include an internal carrier inside a vacuum chamber, an external carrier outside the vacuum chamber, and a carrier interconnection assembly joining the internal carrier in interconnected relationship relative to the external carrier by a plurality of paired magnetic rollers for movement in common of the internal carrier and external carrier upon movement of the external carrier when the vacuum chamber is configured in the reduced pressure condition.

### CROSS REFERENCE TO RELATED APPLICATIONS

This application claims priority to U.S. Provisional Application 62/924,148 titled “Apparatus for Manual Haptic Stimulation of Male Organ” filed Oct. 21, 2019, which is hereby incorporated by reference in entirety.

### FIELD OF THE INVENTION

The disclosure relates to apparatus for manual stimulation of the male sexual organ of an individual.

### BACKGROUND OF THE INVENTION

Manual stimulation of the male sexual organ of an individual may be performed using a penis stroker sleeve. A penis stroker sleeve typically includes a soft interface material, which may be, for example, a skin safe rubber, silicone, latex, or other elastomer-based compound rubber that contacts skin of the penis. The penis stroker sleeve also may include an outer portion that may be manually grasped and manipulated in a back and forth, cyclical stroking motion. When the soft internal material is moved back and forth across the skin of the penis by manually cycling the outer portion, the penis is stimulated by frictional engagement between the soft internal material and penis. Pressure also may be exerted by the stroker sleeve against the penis, through the soft internal material.

Stimulation of the male sexual organ also may be performed using a penis pump to create negative pressure about the penis, so that blood flow into the penis is increased. Penis pumps require a housing, which may be a tube, that can provide a closed, airtight space relative to an airtight seal, around a portion of the penis. The airtight seal, for example, may be formed between the housing and torso. The airtight space located inside the housing and around the penis may be evacuated by pumping air out of the airtight space through a manual air pump, such as a bulb pump or plunger pump. When the airtight space is evacuated, the penis is exposed to negative pressure and thus stimulated to erection by blood flowing into the penis.

When a penis pump as described hereinabove is used to stimulate the penis, a stroker sleeve cannot be properly used, because in order to maintain the airtight seal, the housing of the penis pump prevents manual access and cyclical stroking movement of the stroker sleeve against the penis by hand. Similarly, when a penis pump is used, the housing of the penis pump prevents manual access and stroking of the penis by direct contact with the hand. For reasons stated above and for other reasons which will become apparent to those skilled in the art upon reading and understanding the present specification, there is a need in the art for improved apparatus and methods for stimulating the male sex organ of an individual.

### BRIEF DESCRIPTION OF THE INVENTION

The above-mentioned shortcomings, disadvantages and problems are addressed herein, as will be understood by those skilled in the art upon reading and studying the following specification. This summary is provided to introduce a selection of concepts in a simplified form that are further described below in more detail in the detailed description of embodiments. This summary is not intended to identify key or essential features of the claimed subject matter. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In an embodiment, apparatus for manual haptic stimulation (hereinafter “stimulation apparatus”) of the male sex organ (“penis”) is provided. Such stimulation apparatus may include a penis pump assembly, traveling carrier assembly, and penis stroker sleeve. The penis pump assembly may include a penis pump housing, such as a tubular housing, configured to define an internal penis chamber which may be brought into an airtight condition. The penis pump assembly may include a pump line assembly in fluid communication between the penis chamber and exterior. The pump line assembly may be selectively operable to pump air out of the penis chamber to bring same into the airtight condition, to evacuate the penis chamber and thus create a negative pressure in the penis chamber in the airtight condition. The penis chamber may have a longitudinal axis extending from a proximal end to a distal end opposite the same.

Stimulation apparatus may include a traveling carrier assembly supported relative to the penis pump housing. The traveling carrier assembly may be supported for translation movement relative to the penis pump housing, relative to a longitudinal axis of a penis chamber. Particularly, the travelling carrier assembly may be supported for repeating, cyclical translation movement relative to the penis chamber along the longitudinal axis thereof, through repeating in and out cycles. The traveling carrier assembly may include an internal carrier supported relative to the penis pump housing wall for translation movement in the penis chamber along the longitudinal axis thereof. The internal carrier may include an internal carrier body having a tubular internal carrier wall defining an internal payload space configured to receive and carry a stroker sleeve for translation movement in common with the internal carrier. The internal carrier may include a spaced plurality of internal magnetic rollers configured for rolling motion across the penis pump housing wall interior surface to support the internal carrier body for translation movement relative to the penis pump housing wall and defining a minor internal clearance gap therebetween. The plurality of internal magnetic rollers may be supported in spaced, interconnected relationship by an open mesh. The open mesh may be formed by cooperation of an internal set of elastic fiber members joined in regular, intersecting relationship. The internal carrier body may include a spaced plurality of internal magnetic roller apertures each defined in the internal carrier wall in alignment with a corresponding internal magnetic roller.

The traveling carrier assembly may include an external carrier supported relative to the penis pump housing wall for translation movement outside the housing wall along the longitudinal axis of the penis chamber in common with the internal carrier located inside the penis chamber. The external carrier may include an external carrier body having a tubular external carrier wall defining an external payload space configured to receive the penis pump housing wall for translation movement of the external carrier outside the housing wall along the longitudinal axis of the penis chamber in common with the internal carrier located inside the penis chamber. The external carrier may include a spaced plurality of external magnetic rollers configured for rolling motion across the penis pump housing wall exterior surface to support the external carrier body for translation movement relative to the penis pump housing wall and defining a minor external clearance gap therebetween. The plurality of external magnetic rollers may be supported in spaced, interconnected relationship by an external open mesh. The external open mesh may be formed by cooperation of an external set of elastic fiber members joined in regular, intersecting relationship. The external carrier body may include a spaced plurality of external magnetic roller apertures each defined in the external carrier wall in alignment with a corresponding external magnetic roller.

The traveling carrier assembly may include a carrier interconnection assembly. In an embodiment, for example, the carrier interconnection assembly may include a magnetic carrier interconnection assembly. In an embodiment, for example, a magnetic carrier interconnection assembly may function by magnetism or magnetic connections formed between a spaced plurality of paired magnetic elements. In an embodiment, the carrier interconnection assembly may include a spaced plurality of paired magnetic rollers configured to form by cooperation therebetween a releasable magnetic connection between the internal carrier and external carrier. The plurality of paired magnetic rollers may include a plurality of internal magnetic roller members joined to the internal carrier. The plurality of paired magnetic rollers may include a plurality of external magnetic roller members joined to the external carrier. The plurality of internal magnetic roller members joined to the internal carrier may be selectively aligned to form the plurality of paired magnetic connections in magnetic coupled relationship with the plurality of external magnetic roller members joined to the external carrier, so as to join the internal carrier in interconnected relationship with the external carrier. The plurality of internal magnetic roller members may be selectively misaligned or moved away from the aligned positions, to release the plurality of internal magnetic roller members from the plurality of paired magnetic connections and thus decouple from magnetic relationships with the plurality of external magnetic roller members, so as to release the internal carrier from interconnected relationship with the external carrier.

Apparatus, systems, and methods of varying scope are described herein. These aspects are indicative of various non-limiting ways in which the disclosed subject matter may be utilized, all of which are intended to be within the scope of the disclosed subject matter. In addition to the aspects and advantages described in this summary, further aspects, features, and advantages will become apparent by reference to the associated drawings, detailed description, and claims.

### BRIEF DESCRIPTION OF THE DRAWINGS

The disclosed subject matter itself, as well as further objectives, and advantages thereof, will best be illustrated by reference to the following detailed description of embodiments of the device read in conjunction with the accompanying drawings, wherein:

FIG. 1 is a simplified perspective view of stimulation apparatus in an embodiment, showing the travelling carrier assembly at a proximal position relative to the penis pump housing.

FIG. 2 is a simplified longitudinal cross-section view of the stimulation apparatus shown in FIG. 1.

FIG. 3 is a simplified lateral cross-section view of the stimulation apparatus shown in FIG. 1.

FIG. 4 is a simplified longitudinal cross-section view of the stimulation apparatus, similar to FIG. 2, showing the travelling carrier assembly at a distal position relative to the penis pump housing.

FIG. 5 is an enlarged partial cross-section view of the stimulation apparatus shown generally in FIG. 2, showing detail of the traveling carrier assembly.

### DETAILED DESCRIPTION OF THE INVENTION

In the following detailed description, reference is made to the accompanying drawings which form a part hereof, and in which is shown by way of illustration specific embodiments which may be practiced. These embodiments are described in sufficient detail to enable those skilled in the art to practice the embodiments and disclosure. It is to be understood that other embodiments may be utilized, and that logical, mechanical, electrical, and other changes may be made without departing from the scope of the embodiments and disclosure. In view of the foregoing, the following detailed description is not to be taken as limiting the scope of the embodiments or disclosure.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

The detailed description set forth herein in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

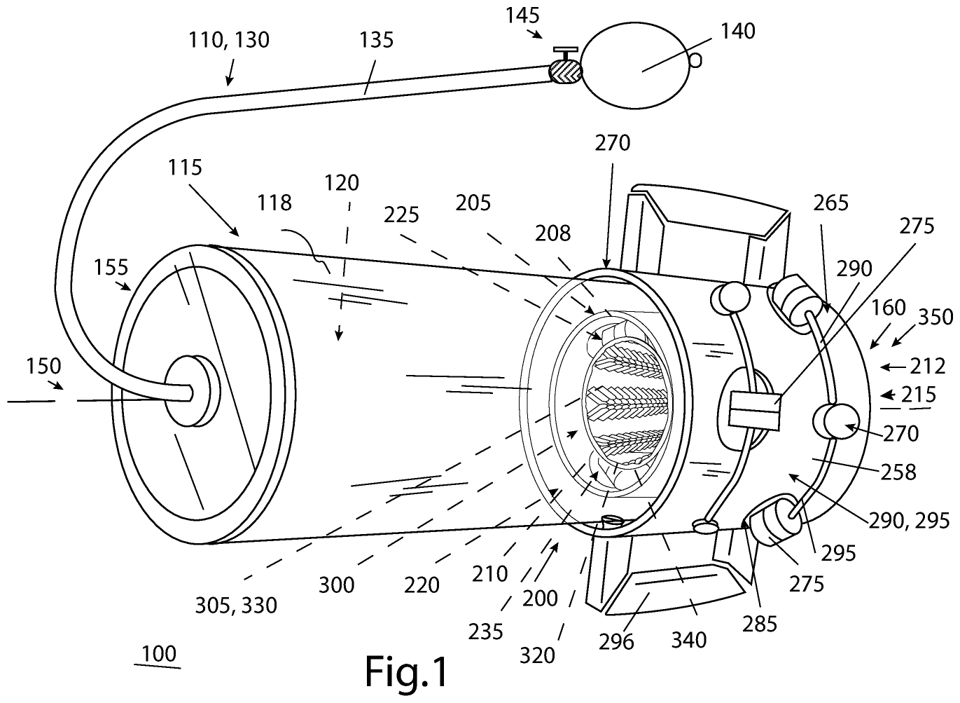

FIG. 1 is a simplified perspective view of stimulation apparatus 100 in an embodiment, showing the traveling carrier assembly 200 at a proximal position relative to the penis pump housing 115. Stimulation apparatus 100 is configured for manual haptic stimulation of the male sex organ (“penis”) 102 of an individual. Stimulation apparatus 100 may include a penis pump assembly 110, traveling carrier assembly 200, and penis stroker sleeve (“stroker sleeve”) 300. The stroker sleeve 300 is configured to receive the penis 102 when inserted therein and form a stroker-to-penis interface with the skin of the penis 102. It will be understood that the stroker sleeve 300 may include soft interface material, as described hereinabove.

As shown in FIG. 1, the penis pump assembly 110 may include a penis pump housing 115, such as a tubular housing. The penis pump housing 115 may be configured to define an internal penis chamber 120 which may be brought into an airtight condition. The penis pump assembly 110 may include a pump line assembly 130 in fluid communication between the penis chamber 120 and exterior environment. The pump line assembly 130 may include pump element 140 joined to pump line 135 and valve 145 operable to open and close air flow through the pump line assembly 130. The pump line assembly 130 may be selectively operable to pump air out of the penis chamber 120 and thus bring the penis chamber 120 into the airtight condition. Operating the pump line assembly 130 may evacuate air from the penis chamber 120 and thus may create a negative pressure in the penis chamber 120 in the airtight condition. The penis chamber 120 may have a longitudinal axis 150 extending from an open proximal end 160 at the torso to a distal end 155 opposite the same.

Stimulation apparatus 100 may include a traveling carrier assembly 200 supported for movement relative to the penis pump housing 115. The traveling carrier assembly 200 may be supported for translation movement relative to the penis pump housing 115, relative to the longitudinal axis 150 of the penis chamber 120. Particularly, the travelling carrier assembly 200 may be supported for repeating, cyclical translation movement relative to the penis chamber 120 along the longitudinal axis 150 thereof, through repeating in and out cycles between a proximal position adjacent the open proximal end 160 and torso, and a distal position adjacent the distal end 155 spaced apart from the torso and nearer the head of penis 102. The traveling carrier assembly 200 may include an internal carrier 205 inside the penis chamber 120, external carrier 255 outside the penis chamber 120, and carrier interconnection assembly 370 defining a releasable magnetic connection between the internal carrier 205 and external carrier 255.

The traveling carrier assembly 200 may include internal carrier 205 supported relative to the penis pump housing wall 118 for translation movement in the penis chamber 120 along the longitudinal axis 150 thereof. The internal carrier 205 may include an internal carrier body 208 having a tubular internal carrier wall 210 defining an internal payload space 262 configured to receive and carry the stroker sleeve 300 for translation movement in common with the internal carrier 205 relative to penis chamber 120 and penis 102. The internal carrier 205 may include a spaced plurality of internal magnetic rollers 225 configured for rolling motion across the penis pump housing wall 118 interior surface to support the internal carrier body 208 for translation movement relative to the penis pump housing wall 118 and defining a minor internal clearance gap therebetween. In an embodiment, the plurality of internal magnetic rollers 225 may be supported in spaced, interconnected relationship by an internal open mesh 290. The internal open mesh 290 may be formed by cooperation of an internal set of elastic fiber members 295 joined in regular, intersecting relationship. The internal carrier 205 may include a spaced plurality of internal fasteners 270 joined to the internal carrier body 208 and securing the plurality elastic fiber members 295 in fixed locations, with each of the plurality of elastic fiber members 295 extending across a corresponding internal magnetic roller aperture 235 to support an internal magnetic roller 225 in the internal magnetic roller aperture 235 for rotation about an axis of rotation defined by the respective elastic fiber member 295. The internal fasteners 270 may include, for example, architectural bolts, sex bolts, barrel bolts or Chicago screws formed of non-magnetic material such as aluminum. The internal carrier body 208 may include a spaced plurality of internal magnetic roller apertures 235 each defined in the internal carrier wall 210 in alignment with a corresponding internal magnetic roller 225. It will be understood that, in an embodiment (not shown), the internal carrier may include a plurality of internal non-magnetic rollers and a separate set of fixed internal magnetic elements joined to the internal carrier body in stationary locations.

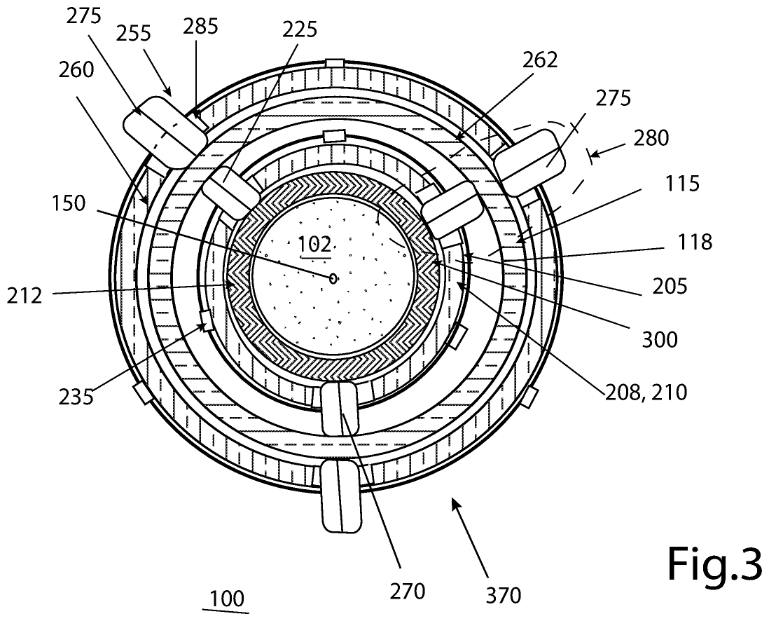

The traveling carrier assembly 200 may include an external carrier 255 supported relative to the penis pump housing wall 118 for translation movement outside the housing wall 118 along the longitudinal axis 150 of penis chamber 120 in common with the internal carrier 205 located inside the penis chamber 120, by force of the magnetic interconnection provided by carrier interconnection assembly 370. The external carrier 255 may include an external carrier body 258 having a tubular external carrier wall 260 defining an external payload space 262 (best shown in FIG. 3) configured to receive the penis pump housing wall 118 for translation movement of the external carrier 255 outside the housing wall 118 along the longitudinal axis 150 of the penis chamber 120 in common with the internal carrier 205 located inside the penis chamber 120. The external carrier 255 may include a spaced plurality of external magnetic rollers 275 configured for rolling motion across the penis pump housing wall 118 exterior surface to support the external carrier body 255 for translation movement relative to the penis pump housing wall 118 and defining a minor external clearance gap therebetween. The plurality of external magnetic rollers 275 may be supported in spaced, interconnected relationship by an external open mesh 290. The external open mesh 290 may be formed by cooperation of an external set of elastic fiber members 295 joined in regular, intersecting relationship. The external carrier 255 may include a spaced plurality of external fasteners 270 joined to the external carrier body 258 and securing the plurality of elastic fiber members 295 in fixed locations, with each of the plurality of elastic fiber members 295 extending across a corresponding external magnetic roller aperture 285 to support an external magnetic roller 275 in the external magnetic roller aperture 285 for rotation about an axis of rotation defined by the respective elastic fiber member 295. The external fasteners 270 may include, for example, architectural bolts, sex bolts, barrel bolts or Chicago screws formed of non-magnetic material such as aluminum. The external carrier body 258 may include a spaced plurality of external magnetic roller apertures 285 each defined in the external carrier wall 260 in alignment with a corresponding external magnetic roller 275. It will be understood that, in an embodiment (not shown), the external carrier may include a plurality of external non-magnetic rollers and a separate set of fixed external magnetic elements joined to the external carrier body in stationary locations. The external carrier 255 may include a set of handles 296 joined to the external carrier body 258 and configured for manual grasping and manipulation by an individual to move the travelling carrier assembly 200 relative to the penis pump housing 115 to cause stroking movement of the stroker sleeve 300 relative to the penis 102.

The traveling carrier assembly 200 may include a carrier interconnection assembly 370. The carrier interconnection assembly 370 may include a spaced plurality of magnetic roller pairs 280. Each magnetic roller pair 280 may include an internal magnetic roller 225 and external magnetic roller 275 configured to form by cooperation therebetween a releasable plurality of magnetic roller pairs 280 defined between the internal carrier 205 and external carrier 255. The plurality of magnetic roller pairs 280 may include a plurality of internal magnetic roller members 225 joined to the internal carrier 205. The plurality of magnetic rollers pairs 280 may include a plurality of external magnetic roller members 275 joined to the external carrier 255. The plurality of internal magnetic roller members 225 joined to the internal carrier 205 may be selectively aligned to form the plurality of magnetic roller pairs 280 in magnetic coupled relationship with the plurality of external magnetic roller members 275 joined to the external carrier 255, so as to join the internal carrier 205 in fixed interconnected relationship with the external carrier 255 without interfering with the airtight seal 125 of housing wall 118 to provide airtight penis chamber 120. The plurality of internal magnetic roller members 225 may be selectively misaligned or moved away from the aligned positions, to release the plurality of internal magnetic roller members 225 from magnetic connection relationship with the plurality of paired external magnetic rollers 275 and thus decouple from magnetic relationships with the plurality of external magnetic roller members 275, so as to release the internal carrier 205 from fixed interconnected relationship with the external carrier 255. It will be understood that the stimulation apparatus 100 may be operable to create and maintain reduced pressure in the airtight penis chamber 120 while the traveling carrier assembly 200 is simultaneously operated to stroke the penis by manual manipulation, by an individual, to move the travelling carrier assembly 200 relative to the penis pump housing 115 to cause stroking movement of the stroker sleeve 300 relative to the penis 102 without disrupting the airtight seal 125.

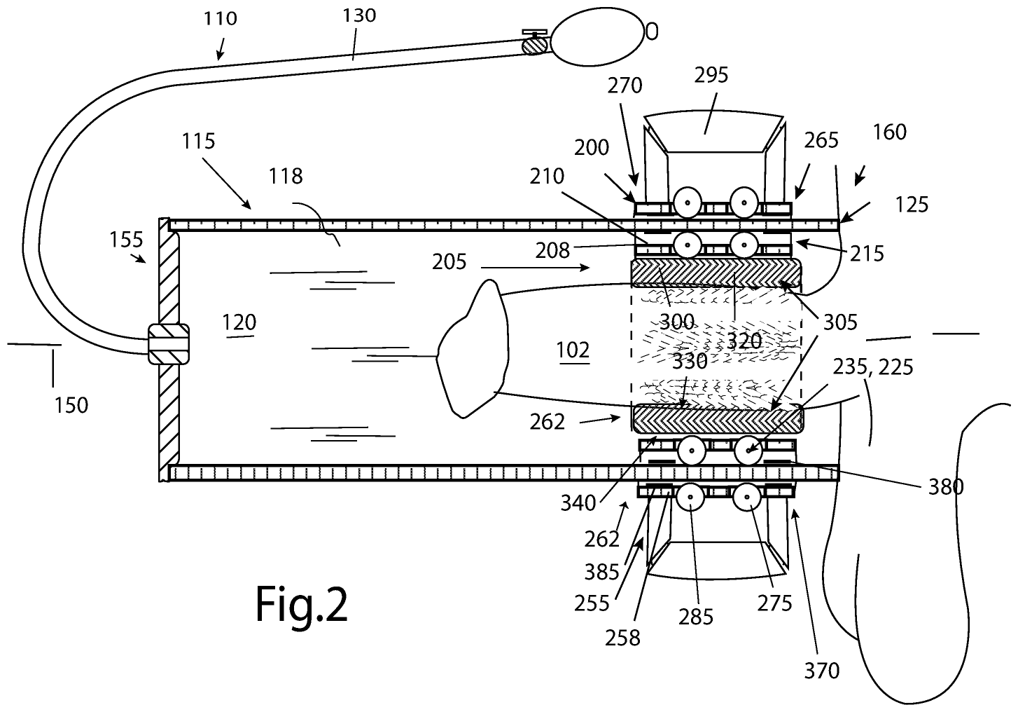

FIG. 2 is a simplified longitudinal cross-section view of the stimulation apparatus 100 shown in FIG. 1. Penis 102 is positioned inside stroker sleeve 300, with the skin in contact with soft interface material 305 thereof.

FIG. 3 is a simplified lateral cross-section view of the stimulation apparatus 100 shown in FIG. 1. The plurality of magnetic roller pairs 280 are shown. Each may include internal magnetic roller 225 in magnetic connection relationship with external magnetic roller 275.

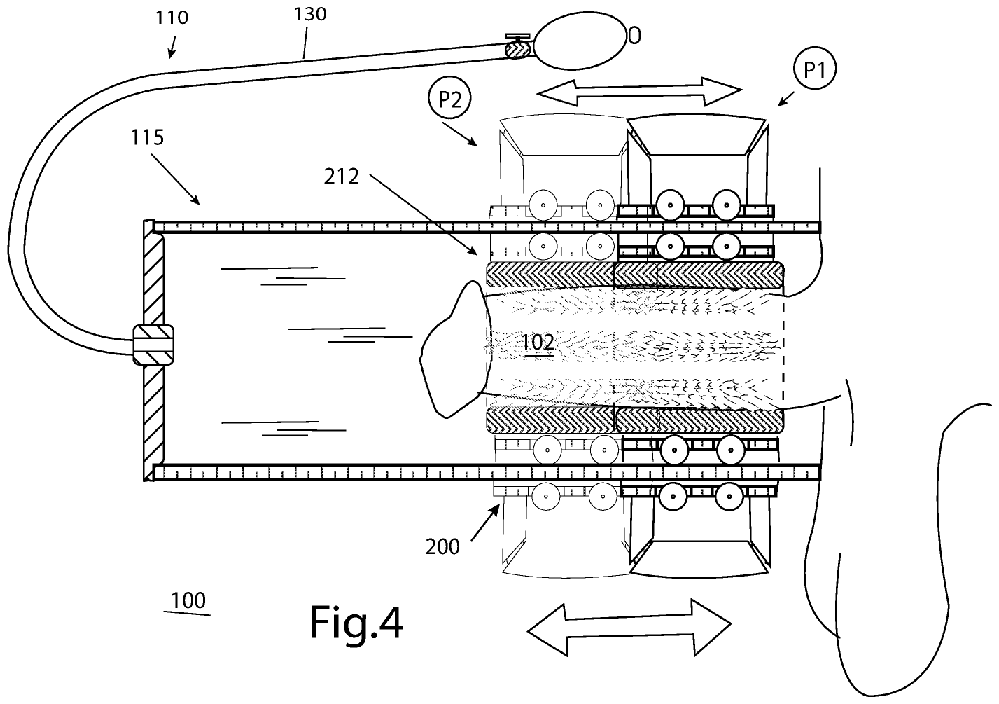

FIG. 4 is a simplified longitudinal cross-section view of the stimulation apparatus 100, similar to FIG. 2, showing the travelling carrier assembly 200 at a distal position relative to the penis pump housing 115. The travelling carrier assembly 200 is shown in first position P1 and second position P2 at different points in stroking movement of penis 102.

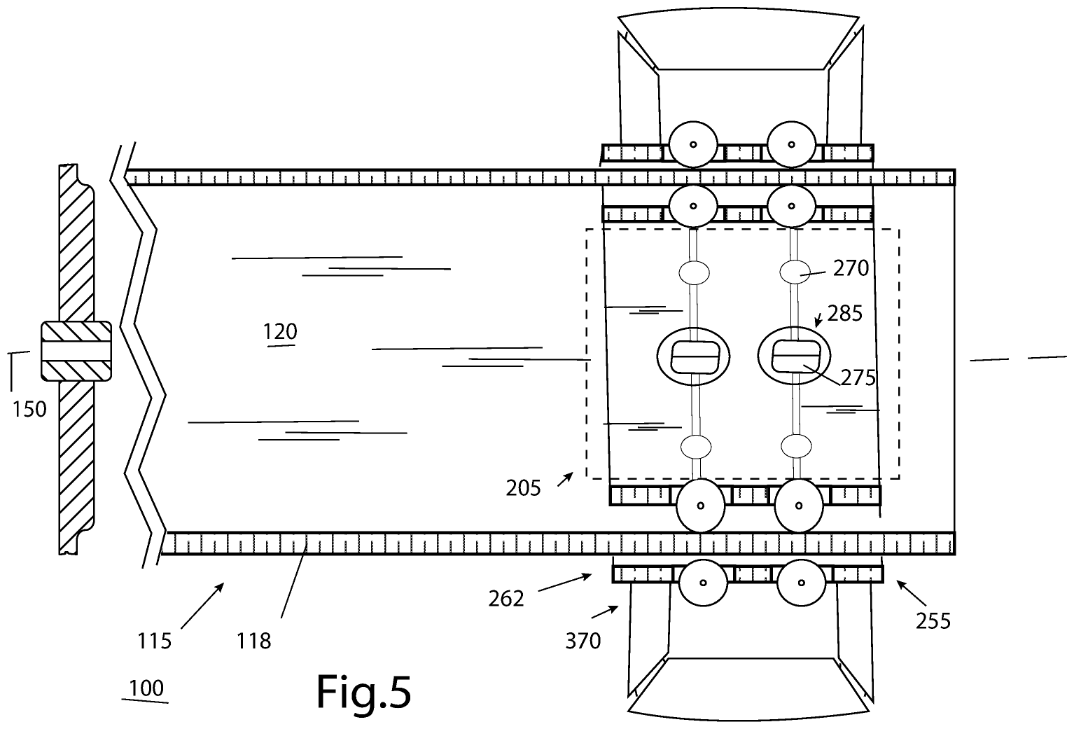

FIG. 5 is an enlarged partial cross-section view of the stimulation apparatus 100 shown generally in FIG. 2, showing detail of the traveling carrier assembly 200. The external magnetic rollers 275 are located in corresponding external magnetic roller apertures 285. The penis is omitted for clarity.

Stimulation apparatus 100 as disclosed is similar in function to a traditional penis pump or a vacuum erectile device (VED) in that it utilizes negative air pressure or a vacuum to enlarge the penis by drawing more of the user's blood into the tissue. A penis pump is used to stretch the tissue and enlarge the overall size of the penis permanently. By leaving the penis in this vacuum chamber for specific amounts of time, the tissues and ligaments in the penis eventually change their size and shape. A vacuum erectile device (VED) utilizes a vacuum in a manner similar to a penis pump, but with the intent of the vacuum erectile device being removed after the vacuum has enlarged the penis. Then an erection ring or penis ring is placed around the base of the penis to restrict the flow of blood from the erect penis. This is done in order to produce a stronger erection or to maintain an erection for a longer period of time. Generally, vacuum erectile devices are prescribed by a physician for self-stimulation or to aid in sexual intercourse for males who have trouble achieving or maintaining an erection naturally.

Stimulation apparatus 100 as disclosed performs an additional, different function different from a penis pump or vacuum erectile device. This different function is a complete and thorough flush of the prostate gland during the vacuum session itself. Much like a regular penis pump or vacuum erectile device, the stimulation apparatus 100 as disclosed comprises a vacuum chamber and an air pump to remove the air and initiate a vacuum around the penis for enlargement. The difference is that instead of wearing the cylinder statically, like a regular penis pump, or using the cylinder temporarily and then removing it once enlargement has occurred, like a vacuum erectile device, the user of stimulation apparatus 100 may leave the penis inside of the vacuum chamber and then stimulate the penis by stroking while maintaining the penis under the full negative air pressure of the vacuum. In the embodiments illustrated, these functions are achieved by an external carrier on the outside of the vacuum cylinder and an internal carrier on the inside vacuum cylinder. The external carrier translates in and out on the outside of the vacuum chamber and functions as a handle for a user to manipulate stroking movement manually. The internal carrier translates in and out on the inside of the vacuum chamber and holds a penis stimulation sleeve, and functions as a stroker carrier manipulated by manual stroking movement of the external carrier. Specifically, in the illustrated embodiments, selected external and internal magnetic rollers which are correctly aligned and positioned are attached to respective of the external carrier and internal carrier, to enable rolling movement along the wall of the vacuum chamber.

Once the user has achieved a “vacuum powered erection,” he may stimulate or masturbate by stroking the penis until achieving orgasm and ejaculation. The vacuum chamber helps the penis become more erect and firmer than would be achieved by way of normal blood pressure in the organ, and thus, the prostate has greater backpressure condition through which to ejaculate seminal fluid than if a lower backpressure condition is created. Once orgasm and ejaculation are achieved, the pubococcygeus muscles convulse and push the seminal fluids through and out of the penis. The vacuum pressure on the penis not only increases rigidity, but makes the penis more sensitive. By bringing more blood into the penis, nerve endings and sensory cells of the penis are pushed closer to the surface of the skin. The result is an extremely expressed prostate gland and a much more intense orgasm than would be achieved without use of the stimulation apparatus.

Apparatus, methods and systems according to embodiments of the disclosure are described. Although specific embodiments are illustrated and described herein, it will be appreciated by those of ordinary skill in the art that any arrangement which is calculated to achieve the same purposes can be substituted for the specific embodiments shown. This application is intended to cover any adaptations or variations of the embodiments and disclosure. For example, although described in terminology and terms common to the field of art, exemplary embodiments, systems, methods and apparatus described herein, one of ordinary skill in the art will appreciate that implementations can be made for other fields of art, systems, apparatus or methods that provide the required functions. The invention should therefore not be limited by the above described embodiment, method, and examples, but by all embodiments and methods within the scope and spirit of the invention. In particular, one of ordinary skill in the art will readily appreciate that the names of the methods and apparatus are not intended to limit embodiments or the disclosure. Furthermore, additional methods, steps, and apparatus can be added to the components, functions can be rearranged among the components, and new components to correspond to future enhancements and physical devices used in embodiments can be introduced without departing from the scope of embodiments and the disclosure. One of skill in the art will readily recognize that embodiments are applicable to future systems, future apparatus, future methods, and different materials. All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope of the disclosure unless otherwise claimed. No language in the specification should be construed as indicating any non-claimed element as essential to the practice of the disclosure as used herein. Terminology used in the present disclosure is intended to include all environments and alternate technologies that provide the same functionality described herein.

What is claimed is:

1. Apparatus for manual haptic stimulation of the male sex organ of an individual, said apparatus comprising:
a penis pump assembly comprising a pump housing, the pump housing comprising a pump housing wall defining a vacuum chamber, the vacuum chamber selectively configurable in a reduced pressure condition, the vacuum chamber configured to receive an inserted penis;
a traveling carrier assembly, the traveling carrier assembly comprising an internal carrier inside the penis chamber, the traveling carrier assembly comprising an external carrier outside the vacuum chamber, the traveling carrier assembly comprising a carrier interconnection assembly joining the internal carrier in interconnected relationship relative to the external carrier, the internal carrier moveable in common with the external carrier upon translation movement of the external carrier, the carrier interconnection assembly functioning across the pump housing wall when the vacuum chamber is configured in the reduced pressure condition.

2. Apparatus according to claim 1, comprising:
a stroker sleeve carried by the internal carrier assembly for translation movement in common with the internal carrier.

3. Apparatus according to claim 1, comprising:
a plurality of internal rollers configured for rolling movement relative to the pump housing wall, the plurality of internal rollers supporting the internal carrier for movement relative to the pump housing wall.

4. Apparatus according to claim 3, comprising:
an internal open mesh configured to support the plurality of internal rollers.

5. Apparatus according to claim 3, comprising:
a plurality of internal magnetic roller apertures defined in the internal carrier to receive the plurality of internal magnetic rollers with clearance to enable rolling movement relative to the internal carrier.

6. Apparatus according to claim 1, comprising:
a plurality of external magnetic rollers configured for rolling movement relative to the pump housing wall, the plurality of external magnetic rollers supporting the external carrier for movement relative to the pump housing wall.

7. Apparatus according to claim 6, comprising:
an external open mesh configured to support the plurality of external magnetic rollers.

8. Apparatus according to claim 6, comprising:
a plurality of external magnetic roller apertures defined in the external carrier to receive the plurality of external magnetic rollers with clearance to enable rolling movement relative to the external carrier.

9. Apparatus according to claim 1, comprising:
the carrier interconnection assembly comprising a plurality of paired magnetic rollers between the internal carrier and external carrier, the plurality of paired magnetic rollers formed between a plurality of internal magnetic rollers mounted to the internal carrier and a plurality of external magnetic rollers mounted to the external carrier, the plurality of internal magnetic rollers aligned with the plurality of external magnetic rollers mounted to the external carrier to establish the plurality of paired magnetic rollers therebetween.

10. Apparatus according to claim 1, comprising:
the penis pump assembly comprising a pump line assembly in fluid communication with the vacuum chamber, the pump line assembly selectively operable to evacuate the vacuum chamber to establish the reduced pressure condition.
